(19) United States
(12) Patent Application Publication
(10) Pub. No.: US 2021/0287766 A1
(43) Pub. Date: Sep. 16, 2021

(54) SYSTEM AND METHOD FOR ANALYSING THE IMAGE OF A POINT-OF-CARE TEST RESULT

(71) Applicants: ACTIM OY, Espoo (FI); AIFORIA TECHNOLOGIES OY, Helsinki (FI)

(72) Inventors: Juuso JUHILA, Helsinki (FI); Tuomas ROPPONEN, Espoo (FI)

(21) Appl. No.: 17/336,425

(22) Filed: Jun. 2, 2021

Related U.S. Application Data
(63) Continuation of application No. PCT/FI2019/050800, filed on Nov. 11, 2019.

(30) Foreign Application Priority Data
Dec. 19, 2018 (FI) 20186112

(51) Int. Cl. G16H 10/40; G16H 30/40; G06T 7/00; G06T 7/90; G06N 3/08

(52) U.S. Cl. CPC G16H 10/40; G16H 30/40; G06T 7/0012; G06T 2207/20084; G06N 3/08; G06T 2207/20081; G06T 7/90

(57) ABSTRACT

A telecommunication network for analyzing a Point-Of-Care, POC, test result includes performing a Point-Of Care, POC, test and getting a test result. A signal from the test result is detected with a camera in a telecommunication terminal and an image is obtained. The image is interpreted by an Artificial Neural Network, ANN, which makes a decision for an analysis of the image. The result of the analysis of the interpreted image is sent to a user interface of an end user. A system for analyzing the result of a point-of-care, POC, test includes a test result of the point-of-care test, a terminal having a camera, and a user interface, and software for interpreting an image of the test result taken by the camera. The software uses an Artificial Neural Network for interpretation of the image and making an analysis.

### TECHNICAL FIELD

The invention is concerned with a method and system for analysing a Point-Of-Care (POC) test result.

### BACKGROUND

Point-Of-Care Testing (POCT), or bedside testing, is generally defined as medical diagnostic testing at or near the point of care at the time and place of patient care instead of sending specimens to a medical laboratory and then waiting hours or days to get the results.

There are several definitions of POCT but no accepted universal definition. Regardless of the exact definition, the most critical elements of POCT are rapid communication of results to guide clinical decisions and completion of testing and follow-up action in the same clinical encounter. Thus, systems for rapid reporting of test results to care providers, and a mechanism to link test results to appropriate counseling and treatment are as important as the technology itself.

The read-out of a POC test result can be assessed by eye or using a dedicated reader for reading the result as an image. The image-analysis algorithms used by such test readers can provides users with qualitative, semi-quantitative and quantitative results.

The algorithms in the test readers used for interpreting Point-Of-Care test results are specifications of how to solve the interpretation of a test result by performing calculation, data processing and automated reasoning tasks. The algorithm could be defined as “a set of rules that precisely defines a sequence of operations”. The algorithms detail the specific instructions a computer should perform in a specific order to carry out the specified task.

Some attempts for developing Artificial Neural Networks (ANNs) for evaluation of test results have been made.

The article “Artificial Neural Network Approach in Laboratory Test Reporting”, learning algorithms by Ferhat Denirci, M D et al, Am J Clin Pathol August 2016, 146:227-237, DOI:10.1093/AJCP/AQW104 is presented as prior art for using algorithms in test reporting based on numerical values. A decision algorithm model by using Artificial Neural Networks (ANNs) is developed on measurement results and can be used to assist specialists in decision making but are not used for direct evaluation of the medical test results.

Computer vision has been proven as a useful tool for quantitative results by measuring the color intensity of the test lines in e.g. lateral flow tests in order to determine the quantity of analyte in the sample. This takes place by capturing and processing test images for obtaining objective color intensity measurements of the test lines with high repeatability.

Solutions for using smartphones to be utilized for lateral flow tests interpretation exist. The article in Sensors 2015, 15, 29569-29593; doi:10.3390/s151129569, “Automated Low-Cost Smartphone-Based Lateral Flow Saliva Test Reader for Drugs-of-Abuse Detection” by Adrian Carrio;*, Carlos Sampedro 1, Jose Luis Sanchez-Lopez 1, Miguel Pimienta 2 and Pascual Campoy presents a smartphone-based automated reader for drug-of-abuse lateral flow assay tests, consisting of a light box and a smartphone device. Test images captured with the smartphone camera are processed in the device using computer vision and machine learning techniques to perform automatic extraction of the results. The development of the algorithm involves segmentation of a test image, where after the regions of interest that represent each segmented strip are preprocessed for obtaining numerical data of the test images before a classification step takes place. Supervised machine learning classifiers based on Artificial Neural Networks (ANN), which is a Multi-Layer Perceptron (MLP), have then been implemented for the classification of the numerical image data.

A smartphone-based colorimetric detection system was developed by Shen et al. (Shen L., Hagen J. A., Papautsky I. Lab Chip. 2012; 12:4240-4243. doi: 10.1039/c2Ic40741h). It is concerned with a point-of-care colorimetric detection with a smartphone together with a calibration technique to compensate for measurement errors due to variability in ambient light.

In the article “Deep Convolutional Neural Networks for Microscopy-Based Point-of care Diagnostics” by John A. Quinn et al. Proceedings of International Conference on Machine Learning for Health Care 2016, JMLR W&C Track Volume 56, presents the use of Convolutional Neural Networks (CNNs) to learn to distinguish the characteristic of pathogens in sample imaging. The training of the model requires annotation of the images with annotation software including e.g. location of pathogens such as plasmodium in thick blood smear images, and tuberculosis bacilli in sputum samples in the form of objects of interest. Upon completion of the CNN, the resulting model is able to classify a small image patch as containing an object of interest or not but requires special selecting of the patches due to identifying overlapping patches.

The efficacy of the immunoassay technology depends on the accurate and sensitive interpretation of spatial features. Therefore, their instrumentation has required fundamental modification and customization to address the technology's evolving needs. The article of 8 May 2015, SPIE newsroom. DOI:10.1117/2.1201504.005861, (Biomedical optics &Medical imaging) (“High-sensitivity, imaging-based immunoassay analysis for mobile applications”) by Onur Mudanyali, Justin White, Chieh-I Chen and Neven Karlovac, presents a reader platform with imaging-based analysis that improves the sensitivity of immunoassay tests used for diagnostics outside the laboratory. The solution includes a smartphone-based reader application for data acquisition and interpretation, test developer software (TDS) for reader configuration and calibration, and a cloud database for tracking of testing results.

### OBJECT OF THE INVENTION

The object of the invention is a fast and portable solution for test result analysis that solves image acquisition problems and accurately interprets point-of-care test results without the need for special readers and advanced image processing.

### Terminology

Neural Networks generally are based on our understanding of the biology of our brains by the structure of the cerebral cortex with the interconnections between the neurons. A perceptron at the basic level is the mathematical representation of a biological neuron. Like in the cerebral cortex, there can be several layers of perceptrons. But, unlike a biological brain where any neuron can in principle connect to any other neuron within a certain physical distance, these artificial neural networks have discrete layers, connections, and directions of data propagation. A perceptron is a linear classifier. It is an algorithm that classifies input by separating two categories with a straight line. The perceptron is a simple algorithm intended to perform binary classification, i.e. it predicts whether input belongs to a certain category of interest or not.

In neural networks, each neuron receives input from some number of locations in the previous layer. In a fully connected layer, each neuron receives input from every element of the previous layer. In a convolutional layer, neurons receive input from only a restricted subarea of the previous layer. So, in a fully connected layer, the receptive field is the entire previous layer. In a convolutional layer, the receptive area is smaller than the entire previous layer.

Deep Learning (also known as deep structured learning or hierarchical learning) differ from conventional machine learning algorithms. The advantage of deep learning algorithms is that they learn high-level features from data in an incremental manner. This eliminates the need of feature extraction required by the conventional task-specific algorithms. Deep learning uses a specific type of algorithm called a Multilayer Neural Network for the learning, which are composed of one input and one output layer, and at least one hidden layer in between. In deep-learning networks, each layer of nodes trains on a distinct set of features based on the previous layer's output.

Artificial Neural Networks (ANN) are neural networks with more than two layers and they are organized in three interconnected layers being the input, the hidden that may include more than one layer, and the output.

A Convolutional Neural Network (CNN) is a class of deep, feed-forward Artificial Neural Networks (ANNs), most commonly applied to analyzing visual imagery. CNNs consist of an input and an output layer, as well as multiple hidden layers.

### SUMMARY OF THE INVENTION

The method of the invention in a telecommunication network for analyzing a Point-Of-Care, POC, test result comprises performing a Point-Of Care, POC, test and getting a test result. A signal from the test result is detected with a camera in a telecommunication terminal and an image is obtained. The image is interpreted by an Artificial Neural Network, ANN, which makes a decision for an analysis of the image. The result of the analysis of the interpreted image is sent to a user interface of an end user.

The system of the invention for analyzing the result of a point-of-care, POC, test comprises a test result of the point-of-care test, a terminal having a camera, and a user interface, and software for interpreting an image of the test result taken by the camera. The software uses an Artificial Neural Network for interpretation of the image and making an analysis.

The preferable embodiments of the invention have the characteristics of the subclaims.

In one such embodiment, the image obtained is sent to a cloud service using the ANN as provided by a service provider belonging to the system. In another one, the image obtained is received by an application in the telecommunication terminal. In the last-mentioned embodiments, the image can be further sent to the cloud service to be interpreted by the ANN in the service provider, the application having access to the cloud service or then the application uses the ANN for the interpretation by software. The analysis of the interpreted image can be sent back to the mobile smart phone and/or a health care institution being the end user(s).

The color balance of the obtained image can be corrected by the application in the telecommunication terminal, wherein software also can select the area of the image for the target of the imaging. The telecommunication terminal can e.g. be a mobile smart phone, a personal computer, a tablet, or a laptop.

The test result is in a visual format and emits a visual signal to be detected by the camera. Alternatively, the signal from the test result is modified into a visual signal by using specific filters.

The Artificial Neural Network, ANN, is trained by deep learning before using it for the interpretation. The training is performed with images in raw format before using the ANN for the analysis of the POC test result. The raw images used for the training can be of different quality with respect to used background, lighting, resonant color, and/or tonal range so that these differences would not affect the interpretation. Also, images from different cameras can be used for the training. In such cases, the Artificial Neural Network, ANN, algorithm can be trained with images labelled with a code indicating the equipment used such as the type and/or model of terminal and/or camera type.

Furthermore, the Artificial Neural Network, ANN, algorithm can take sender information into consideration in the interpretation and has therefore been trained with sender information.

All training images and training data can be stored in a database belonging to the system.

The Artificial Neural Network, ANN, can be a classifier, whereby it can be trained with training data comprising images labelled by classification in pairs of negative or positive results as earlier diagnosed.

The Artificial Neural Network, ANN, can also be a regression model and trained by training data comprising images, which are labelled with percental values for the concentrations of a substance to be tested with the POC test, which percentual values match test results as earlier diagnosed. In this connection, the images can be labelled with normalized values of the percental values, whereby the normalization can be performed by transforming each percentual value to its logarithmic function. Furthermore, the percentual values can be divided into groups and the values of each group are normalized differently.

Furthermore, the Artificial Neural Network, ANN, can be further trained by combining patient data of symptoms with analysis results.

The invention is especially advantageous, when, the Artificial Neural Network, ANN, is a feed-forward artificial neural network, such a s Convolutional Neural Network, CNN. Such a Convolutional Neural Network, CNN, is trained in the invention by and uses semantic segmentation for pointing out the area of interest in the image to be interpreted.

The Artificial Neural Network, ANN, algorithm has preferably also been trained with images labelled with a code indicating the type of used Point-Of-Care, POC, test.

The Point-Of Care, POC, test is especially a flow-through test, a lateral flow test, a drug-screen test, such as a pH or an enzymatic test producing a color or signal that can be detected in the form of a strip with lines, spots, or a pattern, the appearance of which are used for the analysis by the Artificial Neural Network, ANN, in the interpretation of the image of the test result.

The Point-Of Care, POC, can also be a drug-screen test, such as a pH test or an enzymatic test producing a color or signal that can be detected in the form of lines, spots, or a pattern.

The method of the invention is intended for analyzing a point-of-care test result, which is performed by a user on site. An image is taken with a camera from signals emitted from the test result, which can be visual or can be modified to be visual by using specific filters, such as a fluorescence signal or other invisible signal. The camera can be in any terminal such as a mobile device, and preferably a smart phone. The smart phone has preferably an application, that guides a user for taking an image and preferably has access to a cloud service provided by a service provider. The image can in those cases be sent to the service for interpretation. The interpretation is performed by an Artificial Neural Network (ANN), which preferably is a Convolutional neural Network (CNN) and is trained by deep learning in order to be able to perform the interpretation and for making a decision for an analysis of the test result. The analysis can then be sent to a user interface of an end user. The end user can be any of e.g. a patient, a patient data system, a doctor or other data collector.

The system of the invention for analysis of a test result of the point-of-care test (which can be a visual test result) preferably comprises a terminal, such as a mobile device, and preferably a smart phone having a camera, an application which has access to a cloud service, and a user interface, on which the analysis of the interpreted image is shown. It further comprises a service provider with said cloud service providing software for interpreting an image of the test result taken by the camera. The software uses an Artificial Neural Network (ANN) that has been trained by deep learning for interpretation of the image.

In this context, the telecommunication terminal is any device or equipment, which ends a telecommunications link and is the point at which a signal enters and/or leaves a network. Examples of such equipment containing network terminations and are useful in the invention are telephones, such as mobile smart phones and wireless or wired computer terminals, such as network devices, personal computers, laptops, tablets (such as (pads) and workstations. The image can also be scanned and sent to a computer.

In this context, camera stands for any imager, image sensor, image scanner or sensor being able to detect or receive a visual signal, including a visual fluorescence signal, or a signal that can be modified to be visual by using specific filters. Such a filter can be separated from the camera or be built in. Signals that can be modified to be visual includes Ultraviolet (UV), InfraRed (IR), non-visual fluorescence signals and other (like up-converting particles (UCPs). Fluorescence in several wavelengths can also be detected e.g. by an array detector.

Point-Of-Care Testing (POCT) can be thought as a spectrum of technologies, users, and settings from e.g. homes to hospitals. This diversity of Target Product Profiles (TPPs) within POCT is illustrated by the fact that POCT can be done in at least five distinct settings: homes (TPP1), communities (TPP2), clinics (TPP3), peripheral laboratories (TPP4), and hospitals (TPP5). Unique barriers may operate at each level and prevent the adoption and use of POCTs.

In such a framework, the type of device does not define a POC test. POC tests can range from the simplest dipsticks to sophisticated automated molecular tests, portable analysers, and imaging systems. The same lateral flow assay, for example, could be used across all TPPs. Hence, the device does not automatically define the TPP, although some types of devices will immediately rule out some TPPs or users because some devices require a professional or at least a trained user and quality assurance mechanism and restricts the technology to laboratories and hospitals.

Also, the end-user of the test does not automatically define a POC test. The same device (e.g., lateral flow assay), can be performed by several users across the TPPs—from untrained (lay) people, to community health workers, to nurses, to doctors, and laboratory technicians.

Depending on the end-user and the actual setting, the purpose of POC testing may also vary from triage and referral, to diagnosis, treatment, and monitoring.

Anyway, these tests offer rapid results, allowing for timely initiation of appropriate therapy, and/or facilitation of linkages to care and referral. Most importantly, POC tests can be simple enough to be used at the primary care level and in remote settings with no laboratory infrastructure.

POCT is especially used in clinical diagnostics, health monitoring, food safety and environment. It includes e.g. blood glucose testing, blood gas and electrolytes analysis, rapid coagulation testing, rapid cardiac markers diagnostic, drugs of abuse screening, urine protein testing, pregnancy testing, pregnancy monitoring, fecal occult blood analysis, food pathogens screening, hemoglobin diagnostics, infectious disease testing, inflammation state analysis, cholesterol screening, metabolism screening, and many other biomarker analyses.

Thus, POCT is primarily taken from a variety of clinical samples, generally defined as non-infectious human or animal materials including blood, serum, plasma, saliva, excreta (like feces, urine, and sweat), body tissue and tissue fluids (like ascites, vaginal/cervical, amniotic, and spinal fluids).

Examples of Point-Of Care, POC, tests are flow-through tests or lateral flow tests, drug-screen tests, such as a pH or enzymatic tests producing a color or signal that can be detected. POC tests can be used for quantification of one or more analytes.

Flow-through tests or immunoconcentration assays are a type of point of care test in the form of a diagnostic assay that allows users to rapidly test for the presence of a biomarker, usually using a specific antibody, in a sample such as blood, without specialized lab equipment and training. Flow-through tests were one of the first type of immunostrip to be developed, although lateral flow tests have subsequently become the dominant immunostrip point of care device.

Lateral flow tests also known as lateral flow immunochromatographic assays, are the type of point-of-care tests, wherein a simple paper-based device detects the presence (or absence) of a target analyte in liquid sample (matrix) without the need for specialized and costly equipment, though many lab based applications and readers exist that are supported by reading and digital equipment. A widely spread and well-known application is the home pregnancy test.

The fundamental nature of Lateral Flow Assay (LFA) tests relies on the passive flow of fluids through a test strip from one end to the other. A liquid flow of a sample containing an analyte is achieved with the capillary action of porous membranes (such as papers) without external forces.

Commonly, the LF-test consists of a nitrocellulose membrane, an absorption pad, a sample pad and a conjugate pad assembled on a plastic film. Otherwise, this test strip assembly can also be covered by a plastic housing which provides mechanical support. These types of LF-test types and enable liquid flow through the porous materials of the test strip. Currently, the most common detection method of LF-test is based on visual interpretation of color formation on test lines dispensed on the membrane. The color is formed by concentration of colored detection particles (e.g. latex or colloidal gold) in presence of the analyte with no color formed in the absence of the analyte. In regard of some analytes (e.g. small molecules), this assembly can also be vice versa (also called competitive), in which the presence of the analyte is meaning that no color is formed.

The test results are produced in the detection area of the strip. The detection area is the porous membrane (usually composed of nitrocellulose) with specific biological components (mostly antibodies or antigens) immobilized in test and control lines. Their role is to react with the analyte bound to the conjugated antibody. The appearance of those visible lines provides for assessment of test results. The read-out, represented by the lines appearing with different intensities, can be assessed by eye or using a dedicated reader.

Lateral Flow Assay (LFA) based POC devices can be used for both qualitative and quantitative analysis. LF tests are, however, in practice, limited to qualitative or semi-quantitative assays and they may lack the analytical sensitivity, which is needed for detection of many clinically important biomarkers. In addition, a combination of several biomarkers (multiplexing) in the same LF-test has been challenging, because of lack of compatible readers and low analytical sensitivity.

The coupling of POCT devices and electronic medical records enable test results to be shared instantly with care providers.

A qualitative result of a lateral flow assay test is usually based on visual interpretation of the colored areas on the test by a human operator. This may cause subjectivity, the possibility of errors and bias to the test result interpretation.

Although the visually detected assay signal is commonly considered as a strength of LF assays, there is a growing need for simple inexpensive instrumentation to read and interpret the test result.

By just visual interpretation, quantitative results cannot be obtained. These test results are also prone to subjective interpretation, which may lead to unclear or false results. Testing conditions can also affect the visual read-out reliability. For example, in acute situations, the test interpretation may be hindered by poor lighting and movement of objects as well as hurry in acute clinical situations. For this reason, LF-tests based on colored detection particles can be combined with an optical reader that is able to measure the intensity of the color formation on the test.

Thus, hand-held diagnostic devices, known as lateral flow assay readers can provide automated interpretation of the test result. Known automated clinical analyzers, while providing a more reliable result-consistent solution, usually lack portability.

A reader that is detecting visual light enables quantification within a narrow concentration range, but with relatively low analytical sensitivity compared to clinical analyzers. This will rule out detection of some novel biomarkers for which there are high clinical and POC expectations for the future. For this reason, the most important feature of instrument-aided LF-testing is the enhanced test performance; e.g. analytical sensitivity, broader measuring range, precision and accuracy of the quantification. By using other labels (e.g. fluorescent, up-converting or infrared) in LF-assay, more sensitive and quantitative assays can be generated.

A further useful test format for POC in the invention is the microfluidics chip with laboratories on a chip because they allow the integration of many diagnostic tests on a single chip. Microfluidics deal with the flow of liquids inside micrometer-sized channels. Microfluidics study the behavior of fluids in micro-channels in microfluidics devices for applications such as lab-on-a-chip. A microfluidic chip is a set of micro-channels etched or molded into a material (glass, silicon or polymer such as PDMS, for PolyDimethylSiloxane). The micro-channels forming the microfluidic chip are connected together in order to achieve the desired features (mix, pump, sort, or control the biochemical environment). Microfluidics is an additional technology for POC diagnostic devices. There are recent development of microfluidics enabling applications related to lab-on-a-chip.

A lab-on-a-chip (LOC) is a device that integrates one or several laboratory functions on a single integrated circuit (commonly called a “chip”) of only millimeters to a few square centimeters to achieve automation and high-throughput screening. LOCs can handle extremely small fluid volumes down to less than pico-liters. Lab-on-a-chip devices are a subset of microelectromechanical systems (MEMS) devices. However, strictly regarded “lab-on-a-chip” indicates generally the scaling of single or multiple lab processes down to chip-format. Many microfluidistic chips has an area, which is read by a reader as is done in LF-tests.

When the Point-Of Care, POC, test is a flow-through test or a lateral flow test, the test result is given in the form of a strip with colored lines or optionally using spots and/or a pattern. The appearance of these lines, spots, or patterns is the basis for the analysis of the test result itself. The invention uses an Artificial Neural Network (ANN), that has been trained by deep learning for the interpretation of these lines. The Artificial Neural Network (ANN), is preferably a feed-forward artificial neural network, such a s Convolutional Neural Network (CNN).

The invention is especially useful when using the CNN for interpreting the result of a POC lateral flow test since besides qualitative and semi-quantitative results, also quantitative results can be obtained with good accuracy. The invention and obtaining quantitative results are especially useful in connection with rapid cardiac biomarkers, such as Troponin I, Troponin T, Copeptin, CK-MB, D-dimer, FABP3, Galectin-3, Myeloperoxidase, Myoglobin, NT-proBNP & proBNP, Renin, S100B, and ST2 and inflammation state analysis biomarkers, such as AAT, CRP, Calprotectin, IL-6, IL-8, Lactoferrin, NGAL, PCT, Serum Amyloid A, Transferrin, and Trypsinogen-2, especially CRP and calprotectin.

The ANN or CNN is used for the analysis when it has been considered to be trained enough. It is tested against known reference results and when its results are sufficiently accurate, it can be taken for use. The ANN or CNN can, however, be constantly trained by new results for example by linking the analysed test result of a patient to symptoms and thereby learning new relationships for making an analysis. The well-being of users can be presented in different data inquiries, like symptom, health, dietary, sport or other diaries.

Instead of using lines, the test result could be designed to be given in some other form than lines, e.g. the form of a pattern or in the form of spots, such as in the form of a certain pattern of spots.

The ANN or CNN used in the method of the invention can be used for both classification and regression. Classification predicts a label (yes or no) and a regression value predicts a quantity. Thus, the artificial neural network can be a classifier and consists of one or more layers of perceptions indicating a decision of a negative or positive result or then the ANN or CNN is a regression model indicating a decision as a percental value. In classification, the ANN or CNN is trained by images, which are labelled by classification in pairs of negative or positive results as earlier diagnosed. In regression, the ANN or CNN is trained by images, which are labelled with percental values for matching test results as earlier detected or known.

In the annotation, the images can be labelled with a code indicating the used Point-Of-Care, POC, test and/or a code indicating the equipment used such as the type of mobile phone and/or camera type or other type of information, such as the detection time, lot number and test expiration date.

The ANN or CNN algorithm has in preferable embodiments been trained with images from different cameras and/or images of different quality with respect to used background, lighting, resonant color, and/or tonal range.

Image acquisition is an extremely important step in computer vision applications, as the quality of the acquired image will condition all further image processing steps. Images must meet certain requirements in terms of image quality and the relative position of the camera and the object to be captured to enable for the best results. A mobile device is hand-held and, therefore, does not have a fixed position with respect to the test, which is challenging. Furthermore, mobile devices are also used in dynamic environments, implying that ambient illumination has to be considered in order to obtain repeatable results regardless of the illumination conditions.

The color balance of an image may be different in images taken by different cameras and when interpreted by different code readers. A different color balance can also be a consequence of test lot variation. Therefore, in some embodiments of the invention, software in the application of the telecommunication terminal can adjust the intensities of the colors for color correction by some color balance method, such as white balance and QR code correction.

In some embodiments of the invention, software in the application of the telecommunication terminal can also select the area of the image correctly for the target of the imaging.

Not only might the image quality and properties vary. Also, the test equipment, such as the lateral flow strip and test lot variation might vary and have properties leading to images with different properties. The ANN or CNN is also trained for these variances.

The more material the ANN or CNN is trained with, the more accurate it usually is. A training might include a number of e.g. 100 images to 10 000 000 images and from 1 to up to millions of iterations (i.e. training cycles).

In the training, the image to be interpreted is sent to the server.

The ANN or CNN algorithm can also in some embodiments take sender information into consideration in the interpretation.

The interpretation is a result of iteration between different perceptions in the ANN or CNN.

The analysis of the interpreted image is sent back to the telecommunication terminal, such as a mobile smart phone and/or a health care institution, a doctor or other database or end-user as an analysis result.

The system for analyzing the result of a point-of-care test comprises a visual test result of the point-of-care test and a telecommunication terminal, such as a mobile smart phone. The mobile smart phone has a camera, an application having access to a cloud service, and a user interface on which the analysis of the interpreted image is shown. A service provider with a cloud service provides software for interpreting an image of the visual test result taken by the camera. The software uses an artificial neural network algorithm trained with deep learning for being able to interpret the image.

The system further comprises a database with training data of images and image pairs labelled as positive and negative results as diagnosed earlier or images, which are labelled with percental values for matching test results as earlier detected or known. The training data can also involve images from different cameras, backgrounds, and lighting conditions. Furthermore, the training data further comprises information of the camera used, the terminal/smartphone used, and/or the interface.

The advantages of the invention are that it uses deep learning for interpretation of the point-of-care test results and making an analysis on the basis of the interpretation. Conventional machine learning using strict rules has been used for interpretation of the test result images by e.g. classification on images and text, but the invention shows that the deep learning method used performs such tasks even better than actual humans in that it learns to recognize correlations between certain relevant features and optimal results by drawing connections between features.

The invention provides a new approach for analyzing (including quantification) POC test results in being able to train the ANN/CNN directly, preferably using a CNN, with raw images by using deep learning. Raw images are named so because they are not yet processed but contain the information required to produce a viewable image from the camera's sensor data.

In a lateral flow test for classification in accordance with the invention, the training material consists of raw images of test results labelled as positive or negative depending on the appearance of the colored line indicating the test result. The raw images include training material for teaching the ANN/CNN to distinguish between different background colors, light conditions and results from different cameras. For regression, the training material consists of raw images of test results labelled with percentages depending on the intensity of the colored line indicating the test result.

The invention uses semantic segmentation for teaching the ANN/CNN to find the area of interest in the images of the test result. At some point in the analysis, a decision is made about which image points or regions of the image are relevant for further processing. In semantic segmentation each region of an image is labelled in order to partition the image into semantically meaningful parts, and to classify each part into one of the pre-determined classes.

The network used in the invention consists of multiple layers of feature-detecting “perceptions”. Each layer has many neurons that respond to different combinations of inputs from the previous layers. The layers are built up so that the first layer detects a set of primitive patterns in the input, the second layer detects patterns of patterns, the third layer detects patterns of those patterns, and so on. 4 to 1000 distinct layers of pattern recognition are typically used.

Training is performed using a “labelled” dataset of inputs in a wide assortment of representative input patterns that are tagged with their intended output response. In traditional models for pattern recognition, feature extractors are hand designed. In CNNs, the weights of the convolutional layer being used for feature extraction as well as the fully connected layer being used for classification, are determined during the training process. In the CNN used in the invention, the convolution layers play the role of a feature extractor being not hand designed.

Furthermore, the interpreted images can be combined with patient data and further training can be performed by combining symptoms of patients with analysis results of the same patients.

In the following, the invention is described by means of some advantageous embodiments by referring to figures. The invention is not restricted to the details of these embodiments.

### FIGURES

FIG. 1 is an architecture view of a system in which the invention can be implemented

FIG. 2 is a general flow scheme of the method of the invention

FIG. 3 is a flow scheme of a part of the method of the invention, wherein the Artificial Neural Network is trained

FIG. 4 is a test example of the training of a Convolutional Neural Network in accordance with the invention

FIG. 5 is a test example of the performance of the invention

### DETAILED DESCRIPTION

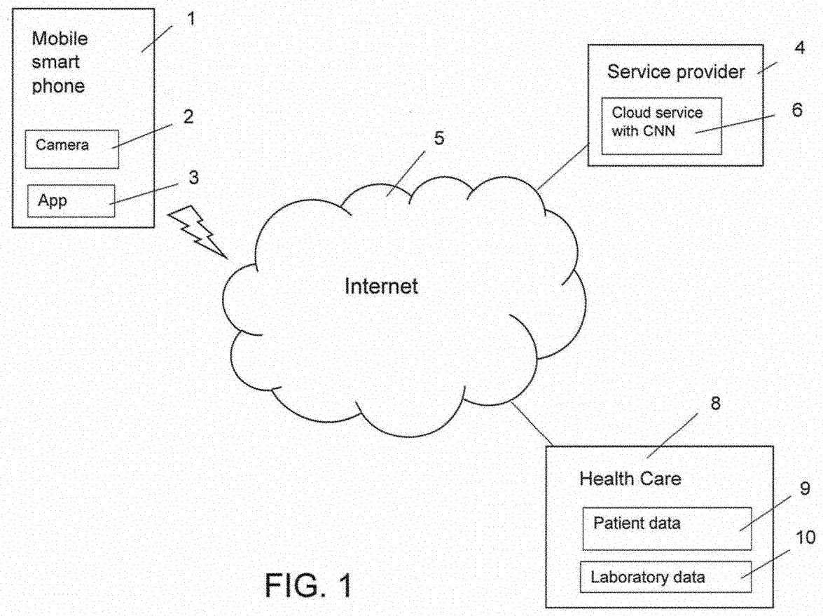

FIG. 1 is an architecture view of a system in which the invention can be implemented.

A mobile smart phone 1 has a camera 2 with which an image of a test result of a Point-Of-Care test can be taken. The image is transferred to an application 3 in the mobile smart phone 1. The application 3 further sends the image to a cloud service provided by a service provider 4 through the Internet 5.

In the cloud service, the image taken is interpreted by an Artificial Neural Network (ANN) 6, which has been trained by deep learning for performing the interpretation of the image for making an analysis. The Artificial Neural Network (ANN) is preferably a Convolutional neural network (CNN).

The analysis of the interpreted image is sent to a user interface of an end user. The end user might be a health care system 8 to which the cloud service is connected via a direct link or through the internet 5. The end user can also be the user of the mobile smart phone 1, whereby the interface can be in the smart phone 1 or can have a link to it. The interface can be in the cloud service, smart phone, and/or in the health care system.

The cloud service can also be connected to a health care system 8 with a patent data system 9 and a laboratory data system 10. The connection can be a direct link or through the internet 5. The interface might have a link to the health care system 8.

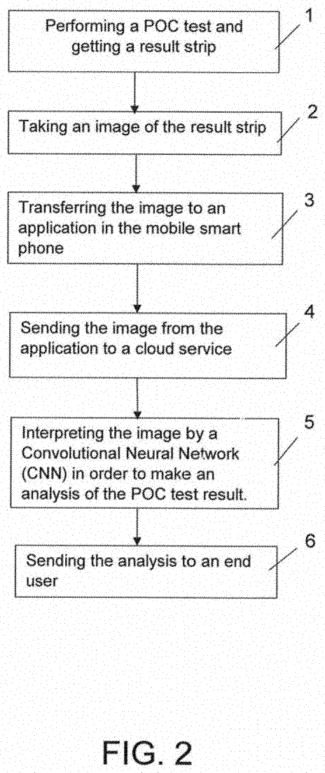

FIG. 2 is a general flow scheme of how the method of the invention can be implemented.

A user performs a Point-Of Care (POC) test is step 1 with a strip on which the result appears with visible lines appearing with different intensities. The appearance of those visible lines is to be analysed. Alternatively, the test result can, instead of lines, consist of specific patterns, lines or spots that necessarily are not visible but can be filtered to be visible by using specific filters.

An image of the test result strip is taken with a camera of a mobile smart phone in step 2.

The image is then transferred to an application in the mobile smart phone in step 3.

In step 4, the image is further sent from the application to a cloud service provided by a service provider.

In step 5, the image is interpreted by the cloud service by using an Artificial Neural Network (ANN), preferably by a Convolutional neural network (CNN), which has been trained with deep learning for the interpretation for making a decision for an analysis of the test result.

In step 6, the analysis of the interpreted image is sent to a user interface of an end user.

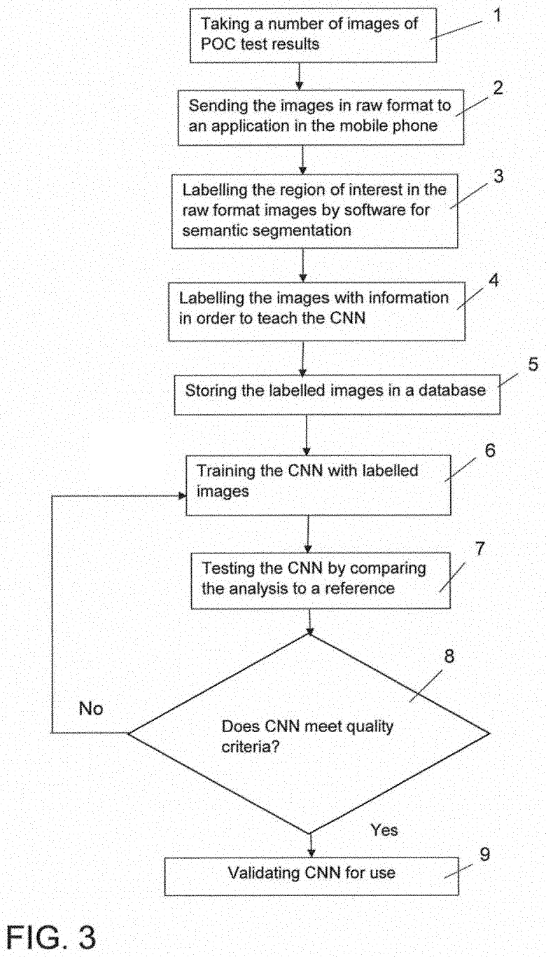

FIG. 3 is a flow scheme of a part of the method of the invention, wherein the Artificial Neural Network (ANN), preferably a Convolutional Neural Network (CNN), used in the invention is trained.

A sufficient number of images of test results of a lateral flow Point-Of-Care test are first taken in step 1 by one or more camera in e.g. a smart phone. The images can thereby have different backgrounds and lighting conditions and the images can be taken with different cameras in different smart phones.

In step 2, sending the images in raw format to an application in the smart phone or to software held by the service.

In step 3, labelling the region of interest in the images of a raw format containing the colored line of the lateral flow test results by software for semantic segmentation by using said images with different backgrounds and lighting conditions and images taken with different cameras in different smart phones.

In step 4, the images are labelled with information in order to teach the Convolutional Neural Network (CNN).

The way of labelling depends on whether the CNN is used for creating a classification model or a regression model.

In classification, the images are labelled in pairs of positive or negative with respect to belonging to a given class by using images with different backgrounds and lighting conditions.

In regression, the images are labelled with percentual values for the concentrations of the substances measured in the POC test. The percentual values match test results as earlier diagnosed. Images with different backgrounds and lighting conditions are preferably used also here.

In some regression embodiments, the percentual values might be normalized by adjusting the values to be used in the labelling in order to get more accurate results. The adjustment can e.g. be performed by logarithmic normalization, wherein each value are transformed into its logarithm function, whereby the concentrations are given in a logarithmic scale. Also other ways of normalization can be performed.

The values can also be divided into a number of different groups on the basis of e.g.

concentration area, for example in four groups, wherein each group of values can be normalized in different ways.

The way of normalization is selected on the basis of the type of POC test.

In step 5, storing the labelled images in a database.

In step 6, training the Convolutional Neural Network (CNN) with the labelled images

In step 7, testing the CNN on a known test result and depending on how the CNN manages, and

either continuing the training with additional training material by repeating step 6 (or all steps 1-6 for getting additional training material)) until the analysis of the results are good enough as compared to a reference test in step 8, or validating the CNN for use in step 9. Criteria is set for evaluating the quality for the comparison.

### Test Example

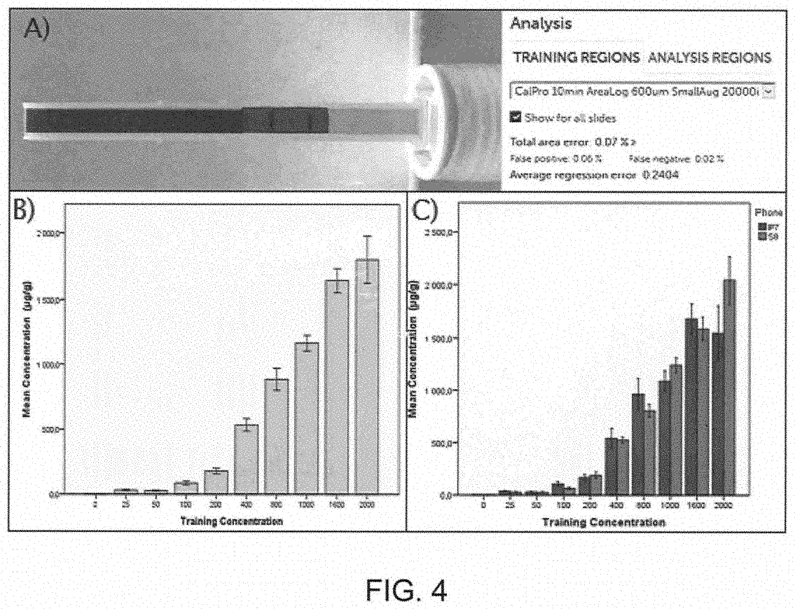

FIG. 4 describes, as an example, the results of the training of a Convolutional Neural Network (CNN) in accordance with the invention.

In, total, 1084 mobile images taken from results of Actim Calprotectin tests were used for CNN training in accordance with the invention. The Actim® Calprotectin test is a lateral flow POC test for the diagnosis of Inflammatory Bowel Diseases, IBD, such as Crohn's disease or ulcerative colitis. The test can be used for semi-quantitative results.

In total, 1084 mobile images taken from results of Actim Calprotectin tests were used for the CNN training. The tests were activated according to the manufacturer's guidelines and photographed by using two mobile cameras; iPhone 7 IP7 and Samsung Galaxy S8;

S8.

The images were transferred to a database, labelled and used for the CNN training. The results are presented in the following:
A) The Analysis region (i.e. detection area) of the Calprotectin tests marked in the middle of the test strip as shown in image A) was found by the CNN after its training with very high statistical confidence The False Positive error being 0.06% and the False Negative error being 0.02%.
   A false positive error being a result indicating the presence of a detection are, where there was no such area, and
   a false negative error being a result missing to indicate an existing detection are, while there in fact was one.
B) Image B shows trained regression values, wherein
   the x-axis shows trained and known Calprotectin concentrations in μg/g) and
   the y-axis shows analysed Calprotectin concentrations in μg/g).
   The trained and known Calprotectin concentrations μg/g) highly correlated with the analysed regression values presented as analysed Calprotectin concentrations in μg/g).
C) Image C shows trained regression values, wherein
   the x-axis shows trained and known Calprotectin concentrations in μg/g) and
   the y-axis shows analysed Calprotectin concentrations in μg/g).
   The columns to the left are results from images taken with a camera in a iPhone 7 IP7 smart phone and the columns to the right are results from images taken with a camera in a Samsung Galaxy S8 smart phone.
   The correlation was similar with both mobile phones used. As conclusions, the trained CNN algorithm shown in here works with a high analytical performance, quantitative behavior, wide detection range and is independent enough of used mobile camera.
   In cases, wherein an even higher accuracy is required, earlier described embodiments of the invention can take performances of different cameras into consideration and make necessary correction, with respect to e.g. color balance.

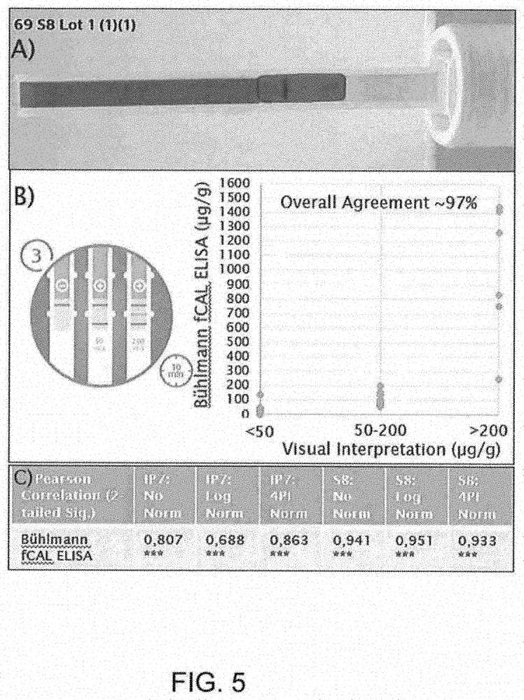

FIG. 5 is a test example of the performance of the invention.

In total 30 stool samples were analysed by using Actim Calprotectin tests according to the manufacturers' instructions.

The Actim Calprotectin test results were interpreted visually and from mobile images by using earlier trained CNN algorithms.

The test results were photographed by using two mobile cameras (iPhone 7; IP7 and Samsung Galaxy S8; S8).

The Mobile images were transferred to the database and then used for CNN analyses.

The performance of the Actim Calprotectin test analysed visually and by CNN was compared with a quantitative Bühlmann fCAL ELISA reference test.

The results are presented in here:

A) The analysis regions of the Calprotectin tests shown in image A) were found after CNN analysis with perfect statistical confidence and there were no detection errors among 30 studied samples.

B) Image B shows a visual interpretation, wherein

the x-axis shows the concentration of calprotectin in μg/g as interpreted visually by Actim Calprotectin; and

the y-axis shows the concentration of calprotectin in μgig as interpreted by the commercial Bühlmann fCAL ELISA test used as a reference test;

the x-axis; Actim Calprotectin in μgig highly correlated (by an overall agreement of ≣96.7%) with the reference test values of the y-axis; Bühlmann fCAL ELISA in μg/g.

C) Image C presents the analysis of the mobile by using CNN training algorithms without normalization (No Norm), with logarithmic normalization (Log Norm) and with area normalization (4PI Norm).

All these analyses showed statistically significant correlation (probability value P<0.001; ***Pearson 2-tailed) when compared to reference test results analysed by Bühlmann fCAL ELISA.

As conclusions, a CNN algorithm trained in accordance with the invention finds the nalytical region (i.e. detection region) of the Actim Calprotectin tests with 100% confidence level. In addition, the Actim Calprotectin test results highly correlated with the Bühlmann reference test, when Actim test is interpreted visually or by using mobile imaging combined with CNN analyses.

1.-33. (canceled)

34. A method in a telecommunication network for analyzing a Point-Of-Care, POC, test result by using an Artificial Neural Network, ANN, that interprets an image of the test result, wherein the Artificial Neural Network, ANN, is a feed-forward Artificial Neural Network, which is a Convolutional Neural Network, CNN, the method comprising:
a) labelling raw format images with areas of interest and with information of earlier diagnosed test results and storing the labelled images in a database,
b) training the Convolutional Neural Network, CNN, with the labelled images,
c) performing a Point-Of Care, POC, test and getting a test result,
d) detecting a signal from the test result with a camera in a telecommunication terminal and obtaining an image,
e) interpreting the image by the Convolutional Neural Network, CNN, which points out an area of interest in the image to be interpreted and makes a decision for an analysis of the image, and
f) sending the result of the analysis of the interpreted image to a user interface of an end user.

35. The method of claim 34, wherein the image obtained in step b) is sent to a cloud service using the ANN as provided by a service provider

36. The method of claim 34, wherein the image obtained in step b) is received by an application in the telecommunication terminal.

37. The method of claim 34, wherein the image obtained in step b) is received by an application in the telecommunication terminal, and the application uses the ANN.

38. The method of claim 37, wherein color balance of the obtained image is corrected by the application.

39. The method of claim 37, wherein software in the application of the telecommunication terminal selects an area of the image for a target of the imaging.

40. The method of claim 34, wherein the telecommunication terminal is a mobile smart phone, a personal computer, a tablet, or a laptop.

41. The method of claim 34, wherein the Point-Of Care, POC, test is a flow-through test or a lateral flow test giving the test result in the form of a strip with a pattern, spots or colored lines, the appearance of which are used for the analysis by the Artificial Neural Network, ANN, in the interpretation of the image of the test result.

42. The method of claim 34, wherein the Point-Of Care, POC, test is a drug-screen test, such as a pH test or an enzymatic test producing a color or signal that can be detected in the form of lines, spots, or a pattern.

43. The method of claim 34, wherein the test result is in a visual format and emits a visual signal to be detected by the camera.

44. The method of claim 34, wherein the signal from the test result consists of specific patterns, lines, or spots that are not visible and are modified into a visual signal by using specific filters.

45. The method of claim 34, wherein the Artificial Neural Network, ANN, algorithm has been trained with raw images of different quality with respect to used background, lighting, resonant color, and/or tonal range.

46. The method of claim 34, wherein the Artificial Neural Network, ANN, algorithm has been trained with images from different cameras.

47. The method of claim 34, wherein the Artificial Neural Network, ANN, algorithm has been trained with images labelled with a code indicating the type of used Point-Of-Care, POC, test.

48. The method of claim 34, wherein the Artificial Neural Network, ANN, algorithm has been trained with images labelled with a code indicating the equipment used such as the type and/or model of terminal and/or camera type.

49. The method of claim 34, wherein the Artificial Neural Network, ANN, is a classifier and is trained by images labelled by classification in pairs of negative or positive results as earlier diagnosed.

50. The method of claim 34, wherein the Artificial Neural Network, ANN, is a regression model and trained by images, which are labelled with percental values for the concentrations of a substance to be tested with the POC test, which percentual values match test results as earlier diagnosed.

51. The method of claim 50, wherein the images are labelled with normalized values of the percental values.

52. The method of claim 51, wherein the normalization is performed by transforming each percentual value to its logarithmic function.

53. The method of claim 51, wherein the percentual values are divided into groups and the values of each group are normalized differently.

54. The method of claim 34, wherein the Artificial Neural Network, ANN, is further trained by combining patient data of symptoms with analysis results.

55. The method of claim 34, wherein the Convolutional Neural Network, CNN, is trained by and uses semantic segmentation for pointing out the area of interest in the image to be interpreted.

56. The method of claim 34, wherein the analysis of the interpreted image is sent back to the mobile smart phone and/or a health care institution being the end user.

57. A system for analyzing the result of a point-of-care, POC, test comprising:
a test result of the point-of-care test,
a database storing raw format images labelled with areas of interest and with information of earlier diagnosed test results,
a terminal having
a camera, and
a user interface,
software for interpreting an image of the test result taken by the camera,
the software using an Artificial Neural Network, ANN, for interpretation of the image by pointing out an area of interest in the image to be interpreted and making a decision for an analysis of the image, wherein the Artificial Neural Network, ANN, is a feed-forward artificial neural network, which is a Convolutional Neural Network, CNN.

58. The system of claim 57, further comprising a service provider with a cloud service providing the software using the Artificial Neural Network, ANN, for interpreting an image of the test result taken by the camera.

59. The system of claim 57, further comprising an application with the software using the Artificial Neural Network, ANN, for interpreting an image of the test result taken by the camera.

60. The system of claim 59, wherein the terminal has an application with access to the cloud service.

61. The system of claim 57, wherein the telecommunication terminal is a mobile smart phone, a personal computer, a tablet, or a laptop.

62. The system of claim 57, wherein the point-of-care test is a flow-through test, a lateral flow test, a drug-screen test, such as a pH or an enzymatic test producing a color or signal that can be detected in the form of a strip with lines, spots, or a pattern, the appearance of which are used for the analysis by the Artificial Neural Network, ANN, in the interpretation of the image of the test result.

63. The system of claim 57, wherein the test result is in a visual format and emits a visual signal to be detected by the camera.

64. The system of claim 57, further comprising one or more specific filters for modifying the test result into a visual signal.

65. The system of claim 57, wherein the Artificial Neural Network, ANN, is a classifier and consists of one or more layers of perceptions indicating a decision of a negative or positive result.

66. The system of claim 57, wherein the Artificial Neural Network, ANN, is a regression model indicating a decision as a percental value.
